(19) United States
(12) Patent Application Publication
(10) Pub. No.: US 2026/0185930 A1
(43) Pub. Date: Jul. 2, 2026

(54) METHOD AND SYSTEM FOR SHORT-TERM FORECASTING AND EARLY WARNING OF WATER QUALITY ALGAL BLOOMS IN LAKES AND RESERVOIRS BASED ON HYPERSPECTRAL PROXIMAL SENSING

(71) Applicant: Nanjing Institute of Geography and Limnology, Chinese Academy of Sciences, Nanjing City (CN)

(72) Inventors: Na LI, Nanjing City (CN); Yunlin ZHANG, Nanjing City (CN); Yibo ZHANG, Nanjing City (CN); Xiayang LUO, Nanjing City (CN)

(21) Appl. No.: 19/428,012

(22) Filed: Dec. 19, 2025

(30) Foreign Application Priority Data
Dec. 26, 2024 (CN) 2024119403224

(51) Int. Cl. G01N 21/31; G01N 33/18; G06F 17/18

(52) U.S. Cl. CPC G01N 21/31; G01N 33/18; G06F 17/18; G01N 2201/1293

(57) ABSTRACT

The present disclosure discloses a method and system for short-term forecasting and early warning of water quality algal blooms in lakes and reservoirs based on hyperspectral proximal sensing, belonging to the technical field of water quality forecasting and early warning methods. The method includes the steps of: combining the preprocessed water proximal hyperspectral reflectance with the spatio-temporal attribute information to generate a water proximal hyperspectral reflectance-time series dataset; combining synchronously measured water quality parameters, and constructing a preset water quality inversion model set according to a spatio-temporal matching principle; generating a multi-water-quality-parameter time series dataset by utilizing the preset water quality inversion model set; building a forecasting and early warning model based on the multi-water-quality-parameter time series dataset; and outputting a water quality algal bloom forecasting and early warning result. The present disclosure transmits measurement data in real time to a cloud server.

### CROSS-REFERENCE TO RELATED APPLICATIONS

This application claims priority to Chinese Patent Application No. 202411940322.4, filed on Dec. 26, 2024, which is incorporated herein by reference in its entirety.

### TECHNICAL FIELD

The present disclosure pertains to the technical field of water environment monitoring and early warning, and specifically relates to a method and system for short-term forecasting and early warning of water quality algal blooms in lakes and reservoirs based on hyperspectral proximal sensing.

### BACKGROUND

High-frequency monitoring and accurate forecasting of water quality and algal blooms serve as crucial foundations and cornerstones for grasping the dynamic characteristics of lake and reservoir water environments, assessing and analyzing change trends, and investigating the causative mechanisms and scientific prevention/control of cyanobacterial blooms. However, traditional manual surveys lag behind the needs of water environment management and decision-making departments in terms of data acquisition frequency, timeliness, and representativeness. Remote sensing via satellites and unmanned aerial vehicles (UAVs) is limited by spatio-temporal and spectral resolution, as well as influences from cloudy/rainy weather and atmospheric correction, proving inadequate for addressing continuous water quality and algal bloom monitoring. High-frequency underwater probes are constrained by high costs, susceptibility to fouling, low accuracy, and calibration difficulties, making it challenging to ensure monitoring accuracy and continuity. Consequently, existing water quality and algal bloom monitoring methods face challenges in capturing rapid ecological disturbances triggered by sudden water pollution incidents or extreme weather events. Furthermore, cyanobacterial blooms result from the coupled effects of physical, chemical, and biological factors in water bodies interacting with meteorological and hydrological factors, involving complex mechanisms and numerous influencing elements. Traditional cyanobacterial bloom forecasting models based on ecological dynamics suffer from issues such as complex formulations, numerous parameters, and partially unclear mechanisms. Statistical models based on water environment data, due to their simple forms and parameter diversity, in addition to poor continuity of input water environment data (primarily relying on daily manual measurements and satellite retrieval data), lead to unsatisfactory forecasting model accuracy, low levels of automation, and limited intelligence.

Aiming at the problems existing in lake and reservoir water quality and algal bloom monitoring and forecasting under the dual influence of current high-intensity human activities and rapid climate change, the present disclosure develops and utilizes an original, minute-level hyperspectral proximal sensing (non-contact, near-ground sensing) water quality instrument. The present disclosure couples multi-scenario measured water quality data, upgrades high-precision inversion models for key water quality parameters in lakes and reservoirs, achieves high-frequency monitoring of water quality and algal blooms under complex weather and water conditions, introduces deep learning algorithms with powerful learning and nonlinear approximation capabilities, constructs a big-data-driven real-time forecasting and early warning model for water quality and algal blooms, facilitates precise prevention and control of cyanobacterial blooms and scientific supervision of the water environment, and improves the intelligence level of ecological environment monitoring and disaster emergency response capabilities.

### SUMMARY

To solve the technical problems existing in the background described above, the present disclosure provides a method and system for short-term forecasting and early warning of water quality algal blooms in lakes and reservoirs based on hyperspectral proximal sensing.

The present disclosure adopts the following technical solution: A method for short-term forecasting and early warning of water quality algal blooms in lakes and reservoirs based on hyperspectral proximal sensing includes the steps of:
collecting water proximal hyperspectral reflectance in real time according to spatio-temporal attribute information, and obtaining preprocessed water proximal hyperspectral reflectance through preprocessing; and combining the preprocessed water proximal hyperspectral reflectance with the spatio-temporal attribute information to generate a water proximal hyperspectral reflectance-time series dataset; wherein the spatio-temporal attribute information at least includes: spatial information, temporal information, and a water body type of a monitoring point;
screening corresponding water proximal hyperspectral reflectance according to the water body type of the monitoring point, combining synchronously measured water quality parameters, and constructing a preset water quality inversion model set related to the water body type of the monitoring point according to a spatio-temporal matching principle; and using the water proximal hyperspectral reflectance-time series dataset as an input variable and the measured water quality parameters as output dependent variables, and generating a multi-water-quality-parameter time series dataset by utilizing the preset water quality inversion model set;
performing statistical analysis at multiple temporal scales based on the multi-water-quality-parameter time series dataset, screening out a key multi-water-quality-parameter time series dataset, and building a forecasting and early warning model; using the preprocessed water proximal hyperspectral reflectance as input features, and outputting a water quality algal bloom forecasting and early warning result; and updating the key multi-water-quality-parameter time series dataset according to the water quality algal bloom forecasting and early warning result, and optimizing the forecasting and early warning model; and
receiving, at a web terminal, user queries for forecasting and early warning demands at different temporal scales, and repeating the above steps to update and display output results in real time.

In a further embodiment, a collection process for collecting the water proximal hyperspectral reflectance includes:

acquiring downwelling irradiance R₁ at a water surface using a transmissive panel with a transmittance of p:

$${R}_{1} = \frac{{R}_{0}}{p} ;$$

where R₀ is irradiance of the transmissive panel; and
acquiring the collected water proximal hyperspectral reflectance R using the formula:

$$R = \frac{{R}_{2}}{{R}_{1}} ;$$

where R₂ is water-leaving radiance of the transmissive panel.

In a further embodiment, preprocessing steps for the preprocessed water proximal hyperspectral reflectance include:
plotting a spectral curve according to the water proximal hyperspectral reflectance, treating the spectral curve as a vector, and calculating a modulus length m₀ of each vector:

$${m}_{0} = \sqrt{{R}_{420}^{2} + {R}_{421}^{2} + {R}_{422}^{2} + … + {R}_{i}^{2}} ;$$

where i∈[420,830], Rᵢ is the water proximal hyperspectral reflectance collected in real time based on the spatio-temporal attribute information;

calculating a ratio of each wavelength to the modulus length using the following formula to obtain a normalized proximal hyperspectral reflectance NRᵢ:

$${NR}_{i} = \frac{{R}_{i}}{{m}_{0}} ;$$

performing linear correction on the normalized proximal hyperspectral reflectance NRᵢ using the following formula:

$${NR}_{i}^{′} = a * {NR}_{i} + b ; where {NR}_{i}^{′}$$

is a radiometrically corrected proximal hyperspectral reflectance, a is a radiometric correction coefficient, and b is a radiometric correction slope.

In a further embodiment, the water body type of the monitoring point at least includes: a clean reservoir, a natural river, an urban river, and a lake;
the synchronously measured water quality parameters at least include: total nitrogen, total phosphorus, chlorophyll-a, transparency, water temperature, dissolved oxygen, permanganate index, turbidity, and total suspended solids concentration; and
the preset water quality inversion model set at least includes: a proximal hyperspectral total phosphorus inversion model, a proximal hyperspectral total nitrogen inversion model, a proximal hyperspectral transparency inversion model, a proximal hyperspectral total suspended solids concentration inversion model, a proximal hyperspectral water temperature inversion model, and a proximal hyperspectral dissolved oxygen inversion model.

In a further embodiment, the spatio-temporal matching principle at least includes: a temporal matching principle and a spatial matching principle;
a representation of the temporal matching principle is: defining a collection time point of the water proximal hyperspectral reflectance as T₀ and a detection time point of the synchronized water quality parameters as T, wherein a relationship between the collection time point T₀ and the detection time point Tis defined as: |T₀−T|≤1 min;

the spatial matching principle includes: a latitude-longitude matching principle and a distance matching principle; a representation of the latitude-longitude matching principle is: M₀=M; where M₀ represents the latitude and longitude of a collection point of the water proximal hyperspectral reflectance, and M represents the latitude and longitude of a detection point of the synchronized water quality parameters; and

a representation of the distance matching principle is: |S₀−S|≤0.1 m; where S₀ is a radius of a standard observation range, and S represents a horizontal distance from a water sample collection point to a monitoring center of a proximal hyperspectral spectrometer; where

$${S}_{0} = H * tan ( \frac{FOV}{2} ) ,$$

H is a height of the proximal hyperspectral spectrometer above a water surface, and FOV represents a field of view angle.

In a further embodiment, the multiple temporal scales at least include: an hourly scale and a daily scale, and the key multi-water-quality-parameter time series dataset includes: hourly water quality parameters and daily water quality parameters; a screening process for the key multi-water-quality-parameter time series dataset includes: introducing correlations W between hourly water quality parameters and synchronized chlorophyll-a concentration, and between daily water quality parameters and synchronized chlorophyll-a concentration, and determining whether a corresponding water quality parameter is a key parameter by assignment of the correlations W:

$$W = \{ \begin{matrix}1 & 0.8 \leq \vert r \vert \leq 1 and p < 0.05 \\ 1 & 0.3 \leq \vert r \vert < 0.8 and p < 0.05 \\ 0 & \vert r \vert < 0.3 or p \geq 0.05\end{matrix} ; where r is a Pearson correlation$$

coefficient, and p is a significance level;
if the assignment of the correlation W is 1, the assignment indicates that the multi-water-quality-parameter exhibits a significant moderate or higher correlation with the synchronized chlorophyll-a concentration, belongs to a key water quality parameter, and is updated into the key multi-water-quality-parameter time series dataset; and if the assignment of the correlation W is 0, the assignment indicates that the multi-water-quality-parameter exhibits a weak correlation with the synchronized chlorophyll-a concentration and does not belong to a key water quality parameter.

In a further embodiment, an analysis process of the forecasting and early warning model includes:

defining a sliding window as L, and a sliding step as d; calculating an average value ω, a standard deviation δ, and a median θ of the multi-water-quality-parameter within an optimal sliding window, respectively, and analyzing a water quality parameter Ψ at a center position of the optimal sliding window:
if Ψ∈[(ω−3)*δ, (ω+3)*δ], the water quality algal bloom forecasting and early warning result is expressed as normal water quality, and no early warning processing is performed; and
if Ψ∉(ω−3)*δ, (ω+3)*δ, the water quality algal bloom forecasting and early warning result is expressed as abnormal water quality, and an alarm message is issued.

In a further embodiment, a matching logical relationship of the spatio-temporal matching principle includes:
assigning a logical judgment result of the spatio-temporal matching principle to Q, assigning Q=1 when the temporal matching principle and the spatial matching principle are simultaneously satisfied, and adding the corresponding synchronously measured water quality parameters and water proximal hyperspectral reflectance to data for model construction; and
conversely, assigning 0 to Q, setting Q=0 when at least one of the temporal matching principle and the spatial matching principle is not satisfied, and deleting the corresponding synchronously measured water quality parameters and water proximal hyperspectral reflectance;

$$Q = \{ \begin{matrix}1 & \vert {T}_{0} - T \vert \leq 1 min ⋂ \vert {S}_{0} - S \vert \leq 0.1 m ⋂ {M}_{0} = M \\ 0 & else\end{matrix} .$$

In a further embodiment, the method further includes: removing the water quality parameter Ψ corresponding to Ψ∉[(ω−3)*δ, (ω+3)*δ] from the key multi-water-quality-parameter time series dataset and replacing the water quality parameter Ψ corresponding to Ψ∉[(ω−3)*δ, (ω+3)*δ] with the median θ; and
performing smoothing processing on the multi-water-quality-parameter time series dataset using a Savitzky-Golay filter to reduce noise during a collection process, performing 5th-order polynomial curve fitting, and performing filling using a nearest neighbor interpolation method, thereby obtaining an updated key multi-water-quality-parameter time series dataset.

A system for short-term forecasting and early warning of water quality algal blooms in lakes and reservoirs based on hyperspectral proximal sensing, configured to implement the aforementioned method for short-term forecasting and early warning of water quality algal blooms, includes:
a first module, configured to collect water proximal hyperspectral reflectance in real time according to spatio-temporal attribute information and obtain preprocessed water proximal hyperspectral reflectance through preprocessing; and combine the preprocessed water proximal hyperspectral reflectance with the spatio-temporal attribute information to generate a water proximal hyperspectral reflectance-time series dataset; wherein the spatio-temporal attribute information at least includes: spatial information, temporal information, and a water body type of a monitoring point;
a second module, configured to screen corresponding water proximal hyperspectral reflectance according to the water body type of the monitoring point, combine synchronously measured water quality parameters, and construct a preset water quality inversion model set related to the water body type of the monitoring point according to a spatio-temporal matching principle; and use the water proximal hyperspectral reflectance-time series dataset as an input variable and the measured water quality parameters as output dependent variables, and generate a multi-water-quality-parameter time series dataset by utilizing the preset water quality inversion model set;
a third module, configured to perform statistical analysis at multiple temporal scales based on the multi-water-quality-parameter time series dataset, screen out a key multi-water-quality-parameter time series dataset, and build a forecasting and early warning model; use the preprocessed water proximal hyperspectral reflectance as input features, and output a water quality algal bloom forecasting and early warning result; and update the key multi-water-quality-parameter time series dataset according to the water quality algal bloom forecasting and early warning result, and optimize the forecasting and early warning model; and
a fourth module, configured to receive, at a web terminal, user queries for forecasting and early warning demands at different temporal scales, and repeat the above steps to update and display output results in real time.

The present disclosure provides the following beneficial effects: The present disclosure uses a proximal hyperspectral spectrometer to achieve real-time, high-frequency acquisition of water body hyperspectral data, and transmits the measurement data in real time to a cloud server for processing. By constructing a water quality inversion model set, hyperspectral reflectance data are converted into water quality parameters (such as total nitrogen, chlorophyll content, dissolved oxygen, etc.) that are easily understandable to users. Based on the multi-parameter water quality data, the present disclosure integrates the data into a software platform.

Users only need to install the software platform on a terminal to view current water quality information, historical long-term changes, and future short-term algal bloom forecasting and early warning information anytime and anywhere, without the need to obtain information through multiple channels or complex procedures. This water quality monitoring method not only improves the efficiency and accuracy of information acquisition but also reduces monitoring costs, expands the monitoring scope, and provides strong technical support for water quality monitoring and environmental protection work.

### BRIEF DESCRIPTION OF THE DRAWINGS

FIG. 1 is a flowchart of a method for short-term forecasting and early warning of water quality algal blooms in lakes and reservoirs based on hyperspectral proximal sensing.

### DETAILED DESCRIPTION OF THE EMBODIMENTS

The following further describes the present disclosure with reference to the accompanying drawings and embodiments.

### Embodiment 1

Cyanobacterial blooms result from the coupled effects of physical, chemical, and biological factors in water bodies interacting with meteorological and hydrological factors, involving complex mechanisms and numerous influencing elements. Traditional cyanobacterial bloom forecasting models based on ecological dynamics suffer from issues such as complex formulations, numerous parameters, and partially unclear mechanisms. Statistical models based on water environment data, due to their simple forms and parameter diversity, in addition to poor continuity of input water environment data, lead to unsatisfactory forecasting model accuracy, low levels of automation, and limited intelligence.

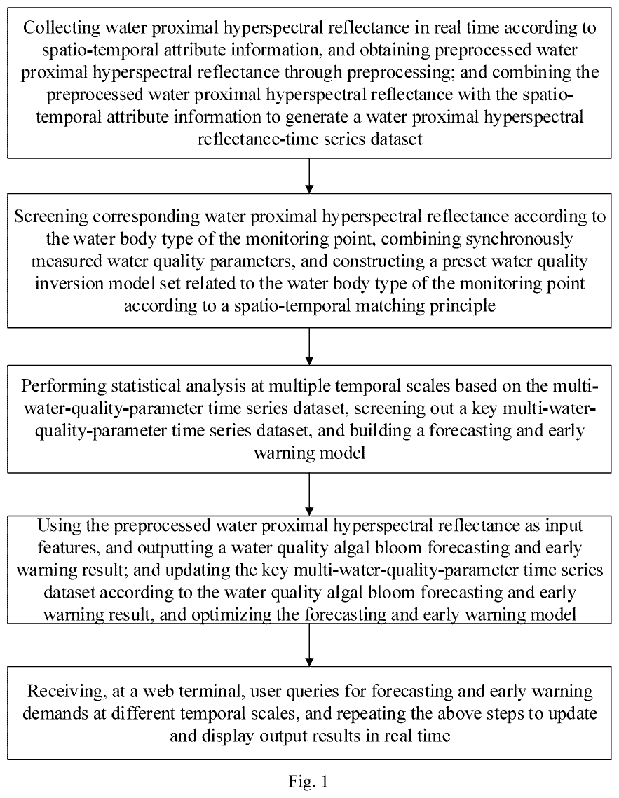

To solve these problems, this embodiment provides a method for short-term forecasting and early warning of water quality algal blooms in lakes and reservoirs based on hyperspectral proximal sensing. As shown in FIG. 1, the method includes the steps of:

Positioning a proximal hyperspectral spectrometer above a water surface based on multiple carrying platforms including ground-based, vessel-based, and tower-based platforms; collecting water proximal hyperspectral reflectance in real time according to spatio-temporal attribute information, and obtaining preprocessed water proximal hyperspectral reflectance through preprocessing; and combining the preprocessed water proximal hyperspectral reflectance with the spatio-temporal attribute information to generate a water proximal hyperspectral reflectance-time series dataset; wherein the spatio-temporal attribute information at least includes: spatial information, temporal information, and a water body type of a monitoring point. In a further embodiment, the spatial information can be understood as geographical longitude and latitude information, and the temporal information can be the year, month, day, hour, minute, and second when the proximal hyperspectral reflectance is acquired. To obtain stable and reliable water proximal hyperspectral reflectance, the installation environment for the proximal hyperspectral spectrometer has strict requirements: 1) no trees, buildings, or shadow obstructions around the equipment; 2) no items within a horizontal range of 2 meters, such as tall white walls or reflective glass, that may cause spectral changes; 3) the water depth in the spectral acquisition area is greater than the water transparency, and the bottom is not visible; 4) no long-term fixed presence of white waves, foam, aquatic vegetation, or other non-water body items within the spectral acquisition area; 5) the lens of the proximal hyperspectral spectrometer should form an angle of 135° to 180° with the plane of the sun; and 6) the proximal hyperspectral spectrometer is positioned at an observation distance of approximately 2 to 10 meters above the water surface.

Screening corresponding water proximal hyperspectral reflectance according to the water body type of the monitoring point, combining synchronously measured water quality parameters, and constructing a preset water quality inversion model set related to the water body type of the monitoring point according to a spatio-temporal matching principle; and using the water proximal hyperspectral reflectance-time series dataset as an input variable and the measured water quality parameters as output dependent variables, and generating a multi-water-quality-parameter time series dataset by utilizing the preset water quality inversion model set.

Performing statistical analysis at multiple temporal scales based on the multi-water-quality-parameter time series dataset, screening out a key multi-water-quality-parameter time series dataset, and building a forecasting and early warning model; using the preprocessed water proximal hyperspectral reflectance as input features, and outputting a water quality algal bloom forecasting and early warning result; and updating the key multi-water-quality-parameter time series dataset according to the water quality algal bloom forecasting and early warning result, and optimizing the forecasting and early warning model.

Receiving, at a web terminal, user queries for forecasting and early warning demands at different temporal scales, and repeating the above steps to update and display output results in real time.

Based on the foregoing description, a collection process for collecting the water proximal hyperspectral reflectance includes:
acquiring downwelling irradiance R₁ at a water surface using a transmissive panel with a transmittance of p:

$${R}_{1} = \frac{{R}_{0}}{p} ;$$

where R₀ is irradiance of the transmissive panel; and
acquiring the collected water proximal hyperspectral reflectance R using the formula:

$$R = \frac{{R}_{2}}{{R}_{1}} ;$$

where R₂ is water-leaving radiance of the transmissive panel.

It is worth mentioning that the proximal hyperspectral spectrometer is inclined at a 45° angle to the horizontal direction, with an azimuth between 90° and 135°, and the lens is equipped with a polarizer to filter out stray light from other environmental directions that is unrelated to water quality, thereby improving the signal-to-noise ratio. The high-definition video camera is a dome camera capable of 360° horizontal rotation and 180° vertical tilt, used to capture and record the current water environment status and query historical water surface conditions, assisting in confirming and judging abnormal water environment situations.

Considering that the apparent optical properties of field water bodies change with lighting conditions and are influenced by various factors such as wind waves, solar elevation angle, and light intensity, preprocessing such as normalization and radiometric correction is required for the proximal hyperspectral water quality reflectance data to eliminate the effects of different weather conditions, wind speeds, and light intensities on the amplitude of spectral curves. The preprocessing at least includes normalization and radiometric correction; the normalization employs a ratio normalization method; the radiometric correction employs a cross-calibration method using a linear function for calibration.

Further, preprocessing steps for the preprocessed water proximal hyperspectral reflectance include:
plotting a spectral curve according to the water proximal hyperspectral reflectance, treating the spectral curve as a vector, and calculating a modulus length m₀ of each vector:

$${m}_{0} = \sqrt{{R}_{420}^{2} + {R}_{421}^{2} + {R}_{422}^{2} + … + {R}_{i}^{2}} ;$$

where i∈[420,830], Rᵢ is the water proximal hyperspectral reflectance collected in real time based on the spatio-temporal attribute information;
calculating a ratio of each wavelength to the modulus length using the following formula to obtain a normalized proximal hyperspectral reflectance NRᵢ:

$${NR}_{i} = \frac{{R}_{i}}{{m}_{0}} ;$$

performing linear correction on the normalized proximal hyperspectral reflectance NRᵢ using the following formula:

$${NR}_{i}^{′} = a * {NR}_{i} + b ; where {NR}_{i}^{′}$$

is a radiometrically corrected proximal hyperspectral reflectance, a is a radiometric correction coefficient, and b is a radiometric correction slope. In this embodiment, the value of a is 1.086, and the value of b is 0.0054.

In another embodiment, the water body type of the monitoring point at least includes: a clean reservoir, a natural river, an urban river, and a lake. Correspondingly, the synchronously measured water quality parameters at least include: total nitrogen, total phosphorus, chlorophyll-a, transparency, water temperature, dissolved oxygen, permanganate index, turbidity, and total suspended solids concentration. Correspondingly, the multi-water-quality-parameter time series dataset is a parameter-time series dataset concerning total nitrogen, total phosphorus, chlorophyll-a, transparency, water temperature, dissolved oxygen, permanganate index, turbidity, and total suspended solids concentration.

The preset water quality inversion model set at least includes: a proximal hyperspectral total phosphorus inversion model, a proximal hyperspectral total nitrogen inversion model, a proximal hyperspectral transparency inversion model, a proximal hyperspectral total suspended solids concentration inversion model, a proximal hyperspectral water temperature inversion model, and a proximal hyperspectral dissolved oxygen inversion model. Among these, the models in the preset water quality inversion model set are constructed by screening water proximal hyperspectral reflectance data for urban rivers, natural rivers, reservoirs, and natural lakes from the water proximal hyperspectral time series dataset based on attribute information, and adding water body attribute type labels; searching for synchronously measured water quality parameters according to spatio-temporal matching principles to generate a quasi-synchronous hyperspectral water quality dataset; following a 3:1 allocation principle, using the hyperspectral data from the generated modeling dataset and validation dataset as input features and the synchronous water quality parameters as output features, and inputting them into a machine learning algorithm to construct a multi-parameter water quality inversion model. The machine learning algorithm in this embodiment may be an Extreme Gradient Boosting (XGBoost) algorithm, support vector machine (SVM), random forest algorithm, deep neural network, or others.

In another embodiment, the spatio-temporal matching principle at least includes: a temporal matching principle and a spatial matching principle;
a representation of the temporal matching principle is: defining a collection time point of the water proximal hyperspectral reflectance as T₀ and a detection time point of the synchronized water quality parameters as T, wherein a relationship between the collection time point T₀ and the detection time point Tis defined as: |T₀−T|≤1 min;
the spatial matching principle includes: a latitude-longitude matching principle and a distance matching principle; a representation of the latitude-longitude matching principle is: M₀=M; where M₀ represents the latitude and longitude of a collection point of the water proximal hyperspectral reflectance, and M represents the latitude and longitude of a detection point of the synchronized water quality parameters; and
a representation of the distance matching principle is: |S₀−S≤0.1 m; where S₀ is a radius of a standard observation range, and S represents a horizontal distance from a water sample collection point to a monitoring center of a proximal hyperspectral spectrometer; where

$${S}_{0} = H * tan ( \frac{FOV}{2} ) ,$$

H is a height of the proximal hyperspectral spectrometer above a water surface, and FOV represents a field of view angle.

Based on the foregoing description, a matching logical relationship of the spatio-temporal matching principle includes:
assigning a logical judgment result of the spatio-temporal matching principle to Q, assigning Q=1 when the temporal matching principle and the spatial matching principle are simultaneously satisfied, and adding the corresponding synchronously measured water quality parameters and water proximal hyperspectral reflectance to data for model construction; and
conversely, assigning 0 to Q, setting Q=0 when at least one of the temporal matching principle and the spatial matching principle is not satisfied, and deleting the corresponding synchronously measured water quality parameters and water proximal hyperspectral reflectance;

$$Q = \{ \begin{matrix}1 & \vert {T}_{0} - T \vert \leq 1 min ⋂ \vert {S}_{0} - S \vert \leq 0.1 m ⋂ {M}_{0} = M \\ 0 & else\end{matrix} .$$

In a further embodiment, the multiple temporal scales at least include: an hourly scale and a daily scale, and the key multi-water-quality-parameter time series dataset includes: hourly water quality parameters and daily water quality parameters. Given that the hourly multi-parameter water quality time series dataset is continuous data, this embodiment employs a 3-sigma criterion for outlier processing, employs median imputation for missing value interpolation, or employs a Savitzky-Golay filter for smoothing to reduce data anomalies and noise caused by factors such as wind waves, solar elevation angle, and sun glint.

A screening process for the key multi-water-quality-parameter time series dataset includes: introducing correlations W between hourly water quality parameters and synchronized chlorophyll-a concentration, and between daily water quality parameters and synchronized chlorophyll-a concentration, and determining whether a corresponding water quality parameter is a key parameter by assignment of the correlations W:

$$W = \{ \begin{matrix}1 & 0.8 \leq \vert r \vert \leq 1 and p < 0.05 \\ 1 & 0.3 \leq \vert r \vert < 0.8 and p < 0.05 \\ 0 & \vert r \vert < 0.3 or p \geq 0.05\end{matrix} ;$$

where r is a Pearson correlation coefficient, and p is a significance level;

if the assignment of the correlation W is 1, the assignment indicates that the multi-water-quality-parameter exhibits a significant moderate or higher correlation with the synchronized chlorophyll-a concentration, belongs to a key water quality parameter, and is updated into the key multi-water-quality-parameter time series dataset; and if the assignment of the correlation Wis 0, the assignment indicates that the multi-water-quality-parameter exhibits a weak correlation with the synchronized chlorophyll-a concentration and does not belong to a key water quality parameter.

It should be noted that, since the growth and reproduction of planktonic algae are closely related to the water environment, the relationships between transparency, total nitrogen, total phosphorus, permanganate index, and temperature obtained through inversion of proximal hyperspectral data and chlorophyll-a were analyzed. The results show that both daily and hourly water quality parameters are extremely significantly correlated with synchronous chlorophyll-a, with the highest correlation observed with the permanganate index, followed by total nitrogen, and then total phosphorus. However, whether for hourly or daily parameters, the correlation between chlorophyll-a concentration and the other inverted water quality parameters is greater than 0.3. This result establishes the correlation between water quality parameters and synchronized chlorophyll-a concentration. Based on the screening of the key multi-water-quality-parameter time series dataset described above, an analysis process of the forecasting and early warning model includes:
defining a sliding window as L, and a sliding step as d, for example, L=11, d=1. calculating an average value ω, a standard deviation δ, and a median θ of the multi-water-quality-parameter within an optimal sliding window, respectively, and analyzing a water quality parameter Ψ at a center position of the optimal sliding window:
if Ψ∈[(ω−3)*δ, (ω+3)*δ], the water quality algal bloom forecasting and early warning result is expressed as normal water quality, and no early warning processing is performed; and
if Ψ∉[(ω−3)*δ, (ω+3)*δ], the water quality algal bloom forecasting and early warning result is expressed as abnormal water quality, and an alarm message is issued.

Further, the method further includes: removing the water quality parameter Ψ corresponding to Ψ∉[(ω−3)*δ, (ω+3)*δ] from the key multi-water-quality-parameter time series dataset and replacing the water quality parameter Ψ corresponding to Ψ∉[(ω−3)*δ, (ω+3)*δ] with the median θ; and
performing smoothing processing on the multi-water-quality-parameter time series dataset using a Savitzky-Golay filter to reduce noise during a collection process, performing 5th-order polynomial curve fitting, and performing filling using a nearest neighbor interpolation method, thereby obtaining an updated key multi-water-quality-parameter time series dataset.

It is worth mentioning that the forecasting and early warning model in this embodiment is a 4-layer long short-term memory (LSTM) network model, comprising an input layer, a hidden layer with 2 memory modules, and a fully connected output layer. 90% of the input key multi-water-quality-parameter time series dataset is used for modeling, and the remaining 10% of the input key multi-water-quality-parameter time series dataset is used for validation. The key water quality parameter time series within the past time steps, such as total nitrogen, total phosphorus, chlorophyll-a, transparency, suspended solids, permanganate index, turbidity, and extinction coefficient, are used as input features.

The chlorophyll-a concentration time series for the forecasting time steps is used as the output feature. The root mean square error (RMSE) and mean absolute percentage error between the output chlorophyll-a and the measured chlorophyll-a are calculated to evaluate the model's accuracy and robustness. Furthermore, the hyperparameters involved in the LSTM network model are enumerated and compared using a grid search method. The model is evaluated using a 5-fold cross-validation method to screen for the optimal hyperparameters: the numbers of neurons in the 2 LSTM layers are 100 and 150 respectively, the optimizer is Adam, the activation function is Sigmoid, and the number of model iterations is set to 60. The forecasting time steps are for the future 1 hour, 2 hours, 3 hours, as well as 1 day, 2 days, and 3 days.

The past time steps range from 2 to 30. The optimal step size refers to the past time step length when the error of the LSTM network model, enumerated and compared based on past time steps ranging from 2 to 30, is minimized. The forecasting and early warning model at this point is also the optimal model.

The water quality data presented to users by this method are data collected in real-time and updated at the current monitoring point, while this method also provides rapid water quality changes at different temporal scales during historical operation, thereby ensuring users can comprehensively understand current and past water quality change information.

This method supports web-based interaction, allowing users to select query content based on their own needs and focus points. Users can intuitively view the water quality status at points of interest and conveniently access historical water quality time series changes and future short-term algal bloom change information, thereby significantly improving query and monitoring efficiency and enhancing the level of decision support.

This method enables real-time, high-frequency monitoring, display, and forecasting and early warning of water quality information, ensuring users obtain the latest and most accurate information to assist in decision-making. Compared to the information lag inherent in traditional manual monitoring, this method offers higher real-time capability and foresight.

This method displays water quality information of interest to users based on their needs and focus points, meaning users can intuitively obtain effective information without interference from other information.

### Embodiment 2

This embodiment provides a system for short-term forecasting and early warning of water quality algal blooms in lakes and reservoirs based on hyperspectral proximal sensing, configured to implement the method for short-term forecasting and early warning of water quality algal blooms as described in Embodiment 1. The system includes:

a first module, configured to collect water proximal hyperspectral reflectance in real time according to spatio-temporal attribute information and obtain preprocessed water proximal hyperspectral reflectance through preprocessing; and combine the preprocessed water proximal hyperspectral reflectance with the spatio-temporal attribute information to generate a water proximal hyperspectral reflectance-time series dataset; wherein the spatio-temporal attribute information at least includes: spatial information, temporal information, and a water body type of a monitoring point;
a second module, configured to screen corresponding water proximal hyperspectral reflectance according to the water body type of the monitoring point, combine synchronously measured water quality parameters, and construct a preset water quality inversion model set related to the water body type of the monitoring point according to a spatio-temporal matching principle; and use the water proximal hyperspectral reflectance-time series dataset as an input variable and the measured water quality parameters as output dependent variables, and generate a multi-water-quality-parameter time series dataset by utilizing the preset water quality inversion model set;
a third module, configured to perform statistical analysis at multiple temporal scales based on the multi-water-quality-parameter time series dataset, screen out a key multi-water-quality-parameter time series dataset, and build a forecasting and early warning model; use the preprocessed water proximal hyperspectral reflectance as input features, and output a water quality algal bloom forecasting and early warning result; and update the key multi-water-quality-parameter time series dataset according to the water quality algal bloom forecasting and early warning result, and optimize the forecasting and early warning model; and
a fourth module, configured to receive, at a web terminal, user queries for forecasting and early warning demands at different temporal scales, and repeat the above steps to update and display output results in real time.

What is claimed is:

1. A method for short-term forecasting and early warning of water quality algal blooms in lakes and reservoirs based on hyperspectral proximal sensing, comprising the steps of:
collecting water proximal hyperspectral reflectance in real time according to spatio-temporal attribute information, and obtaining preprocessed water proximal hyperspectral reflectance through preprocessing; and combining the preprocessed water proximal hyperspectral reflectance with the spatio-temporal attribute information to generate a water proximal hyperspectral reflectance-time series dataset; wherein the spatio-temporal attribute information comprises: spatial information, temporal information, and a water body type of a monitoring point;
screening corresponding water proximal hyperspectral reflectance according to the water body type of the monitoring point, combining synchronously measured water quality parameters, and constructing a preset water quality inversion model set related to the water body type of the monitoring point according to a spatio-temporal matching principle; and using the water proximal hyperspectral reflectance-time series dataset as an input variable and the measured water quality parameters as output dependent variables, and generating a multi-water-quality-parameter time series dataset by utilizing the preset water quality inversion model set;
performing statistical analysis at multiple temporal scales based on the multi-water-quality-parameter time series dataset, screening out a key multi-water-quality-parameter time series dataset, and building a forecasting and early warning model; using the preprocessed water proximal hyperspectral reflectance as input features, and outputting a water quality algal bloom forecasting and early warning result; and updating the key multi-water-quality-parameter time series dataset according to the water quality algal bloom forecasting and early warning result, and optimizing the forecasting and early warning model;
wherein the multiple temporal scales comprise: an hourly scale and a daily scale, and the key multi-water-quality-parameter time series dataset comprises: hourly water quality parameters and daily water quality parameters; a screening process for the key multi-water-quality-parameter time series dataset comprises: introducing correlations W between hourly water quality parameters and synchronized chlorophyll-a concentration, and between daily water quality parameters and synchronized chlorophyll-a concentration, and determining whether a corresponding water quality parameter is a key parameter by assignment of the correlations W:
$$W = \{ \begin{matrix}1 & 0.8 \leq \vert r \vert \leq 1 and p < 0.05 \\ 1 & 0.3 \leq \vert r \vert < 0.8 and p < 0.05 \\ 0 & \vert r \vert < 0.3 or p \geq 0.05\end{matrix} ;$$
where r is a Pearson correlation coefficient, and p is a significance level;
if the assignment of the correlation Wis 1, the assignment indicates that the multi-water-quality-parameter exhibits a significant moderate or higher correlation with the synchronized chlorophyll-a concentration, belongs to a key water quality parameter, and is updated into the key multi-water-quality-parameter time series dataset; if the assignment of the correlation W is 0, the assignment indicates that the multi-water-quality-parameter exhibits a weak correlation with the synchronized chlorophyll-a concentration and does not belong to a key water quality parameter; and receiving, at a web terminal, user queries for forecasting and early warning demands at different temporal scales, and repeating the above steps to update and display output results in real time;
wherein an analysis process of the forecasting and early warning model comprises:
defining a sliding window as L, and a sliding step as d; calculating an average value ω, a standard deviation δ, and a median θ of the multi-water-quality-parameter within an optimal sliding window, respectively, and analyzing a water quality parameter Ψ at a center position of the optimal sliding window:
if Ψ∈[(ω−3)*δ, (ω+3)*δ], the water quality algal bloom forecasting and early warning result is expressed as normal water quality, and no early warning processing is performed;
if Ψ∉[(ω−3)*δ, (ω+3)*δ], the water quality algal bloom forecasting and early warning result is expressed as abnormal water quality, and an alarm message is issued;
the method further comprises: removing the water quality parameter Ψ corresponding to Ψ∉[(ω−3)*δ, (ω+3)*δ] from the key multi-water-quality-parameter time series dataset and replacing the water quality parameter Ψ corresponding to Ψ∉[(ω−3)*δ, (ω+3)*δ] with the median θ; and
performing smoothing processing on the multi-water-quality-parameter time series dataset using a Savitzky-Golay filter to reduce noise during a collection process, performing 5th-order polynomial curve fitting, and performing filling using a nearest neighbor interpolation method, thereby obtaining an updated key multi-water-quality-parameter time series dataset.

2. The method for short-term forecasting and early warning of water quality algal blooms in lakes and reservoirs based on hyperspectral proximal sensing according to claim 1, wherein a collection process for collecting the water proximal hyperspectral reflectance comprises:
acquiring downwelling irradiance R₁ at a water surface using a transmissive panel with a transmittance of p:
$${R}_{1} = \frac{{R}_{0}}{p} ;$$
where R₀ is irradiance of the transmissive panel; and
acquiring the collected water proximal hyperspectral reflectance R using the formula:
$$R = \frac{{R}_{2}}{{R}_{1}} ;$$
where R₂ is water-leaving radiance of the transmissive panel.

3. The method for short-term forecasting and early warning of water quality algal blooms in lakes and reservoirs based on hyperspectral proximal sensing according to claim 1, wherein preprocessing steps for the preprocessed water proximal hyperspectral reflectance comprise:
plotting a spectral curve according to the water proximal hyperspectral reflectance, treating the spectral curve as a vector, and calculating a modulus length m₀ of each vector:
$${m}_{0} = \sqrt{{R}_{420}^{2} + {R}_{421}^{2} + {R}_{422}^{2} + … + {R}_{i}^{2}} ;$$
where i∈[420,830], Rᵢ is the water proximal hyperspectral reflectance collected in real time based on the spatio-temporal attribute information;
calculating a ratio of each wavelength to the modulus length using the following formula to obtain a normalized proximal hyperspectral reflectance NRᵢ:
$${NR}_{i} = \frac{{R}_{i}}{{m}_{0}} ;$$
performing linear correction on the normalized proximal hyperspectral reflectance NR; using the following formula:
$${NR}_{i}^{′} = a * {NR}_{i} + b ; where {NR}_{i}^{′}$$
hyperspectral reflectance, a is a radiometric correction coefficient, and b is a radiometric correction slope.

4. The method for short-term forecasting and early warning of water quality algal blooms in lakes and reservoirs based on hyperspectral proximal sensing according to claim 1, wherein the water body type of the monitoring point comprises: a clean reservoir, a natural river, an urban river, and a lake;
the synchronously measured water quality parameters comprise: total nitrogen, total phosphorus, chlorophyll-a, transparency, water temperature, dissolved oxygen, permanganate index, turbidity, and total suspended solids concentration; and
the preset water quality inversion model set comprises: a proximal hyperspectral total phosphorus inversion model, a proximal hyperspectral total nitrogen inversion model, a proximal hyperspectral transparency inversion model, a proximal hyperspectral total suspended solids concentration inversion model, a proximal hyperspectral water temperature inversion model, and a proximal hyperspectral dissolved oxygen inversion model.

5. The method for short-term forecasting and early warning of water quality algal blooms in lakes and reservoirs based on hyperspectral proximal sensing according to claim 1, wherein the spatio-temporal matching principle comprises: a temporal matching principle and a spatial matching principle;
a representation of the temporal matching principle is: defining a collection time point of the water proximal hyperspectral reflectance as T₀ and a detection time point of the synchronized water quality parameters as T, wherein a relationship between the collection time point T₀ and the detection time point T is defined as: |T₀−T≤1 min;
the spatial matching principle comprises: a latitude-longitude matching principle and a distance matching principle; a representation of the latitude-longitude matching principle is: M₀=M; where M₀ represents the latitude and longitude of a collection point of the water proximal hyperspectral reflectance, and M represents the latitude and longitude of a detection point of the synchronized water quality parameters; and
a representation of the distance matching principle is: |S₀−S|≤0.1 m; where S₀ is a radius of a standard observation range, and S represents a horizontal distance from a water sample collection point to a monitoring center of a proximal hyperspectral spectrometer; where
$${S}_{0} = H * tan ( \frac{FOV}{2} ) ,$$
H is a height of the proximal hyperspectral spectrometer above a water surface, and FOV represents a field of view angle.

6. The method for short-term forecasting and early warning of water quality algal blooms in lakes and reservoirs based on hyperspectral proximal sensing according to claim 5, wherein a matching logical relationship of the spatio-temporal matching principle comprises:
assigning a logical judgment result of the spatio-temporal matching principle to Q, assigning Q=1 when the temporal matching principle and the spatial matching principle are simultaneously satisfied, and adding the corresponding synchronously measured water quality parameters and water proximal hyperspectral reflectance to data for model construction; and
conversely, assigning 0 to Q, setting Q=0 when at least one of the temporal matching principle and the spatial matching principle is not satisfied, and deleting the corresponding synchronously measured water quality parameters and water proximal hyperspectral reflectance;
$$Q = \{ \begin{matrix}1 & \vert {T}_{0} - T \vert \leq 1 min ⋂ \vert {S}_{0} - S \vert \leq 0.1 m ⋂ {M}_{0} = M \\ 0 & else\end{matrix} .$$
